(19) United States
(12) Patent Application Publication
(10) Pub. No.: US 2013/0226223 A1
(43) Pub. Date: Aug. 29, 2013

(54) EMBOLIC PROTECTION DEVICES, VASCULAR DELIVERY CATHETERS, AND METHODS OF DEPLOYING SAME

(71) Applicant: Benjamin Spenser, M.P. Hof Karmel (IL)

(72) Inventor: Benjamin Spenser, M.P. Hof Karmel (IL)

(73) Assignee: GARDIA MEDICAL LTD., Caesarea (IL)

(21) Appl. No.: 13/637,023

(22) PCT Filed: Mar. 23, 2011
(86) PCT No.: PCT/IB11/01106

§ 371 Date: May 6, 2013

Related U.S. Application Data
(60) Provisional application No. 61/340,772, filed on Mar. 23, 2010.

(51) Int. Cl. A61F 2/01

(52) U.S. Cl. CPC A61F 2/01
USPC 606/200

(57) ABSTRACT

A method of deploying multiple filtering elements within selected vasculature includes the steps of delivering and deploying a first filtering element over a primary guidewire to a first vessel branching off a main vessel, locking the first filtering element onto the primary guidewire at a desired location within the first branching vessel, delivering a secondary guidewire to a second vessel branching off the main vessel, delivering a second filtering element over the secondary guidewire to the second branching vessel, deploying the second filtering element at a desired location within the second branching vessel, and locking the second filtering element onto the primary guidewire at the desired location associated with the second branching vessel.

### CROSS-REFERENCE TO RELATED APPLICATION

This application claims the benefit of priority of U.S. Provisional Patent Application No. 61/340,772, filed on Mar. 23, 2010, the disclosure of which is incorporated herein by reference.

### TECHNICAL FIELD

The present disclosure relates to embolic protection devices, vascular delivery catheters, and methods of deploying the same. More particularly, the disclosure relates to arterial filters, catheters for delivery and retrieval of arterial filters, and methods of deploying the same for use in catheterizations or other minimally-invasive procedures.

### BACKGROUND

Minimally-invasive endovascular procedures are increasingly used for treatment of various cardiac and peripheral diseases. Various catheter-based procedures for removing vascular blockages associated with plaque (i.e., vascular stenoses) in the cardiovasculature have evolved over the past few decades, including without limitation, angioplasty (PTCA and PTA), stenting, and atherectomy procedures.

During such minimally-invasive procedures, the potential exists for plaque located at the vascular site to become dislodged by the therapeutic catheter (e.g., expandable dilatation balloon, expandable stent, atherectomy device, etc.), which could thereafter embolize to other vascular sites and vital organs. For example, it is possible that procedures involving catheterization of the aorta, and cardiac vasculature or intra-cardiac chambers could produce debris which can embolize into the carotid vessels resulting in stroke or death.

Consequently, various embolic protection devices (e.g., embolic filters) have been introduced for adjunctive use with such therapeutic catheter procedures, which are generally deployed within or near the vascular site being treated, and most typically located distal of the therapeutic site (i.e., downstream with respect to direction of blood flow) in order to filter out and trap any embolic debris generated by the interventional procedure.

More recently, these minimally-invasive techniques have progressed into the area of heart valve repair (e.g., valvuloplasty) and valve replacement of dysfunctional valve structure (e.g., implantation of prosthetic valves for replacement of the native, diseased aortic, mitral, tricuspid or pulmonary valves). Perhaps the most prevalent of these procedures is transcatheter aortic-valve implantation (TAVI), but minimally-invasive techniques have also been developed for repair and replacement of the other heart valves.

Due to the size of the prosthetic heart valve and the delivery catheter required, aortic valve replacement is typically performed by catheterization using the femoral artery approach, namely, traversing the aortic arch to access the native valve (i.e., progressing in the direction from the left atrium to the left ventricle). More recently it has become possible to introduce a replacement aortic valve by exposing the heart in a minimally-invasive manner and entering the heart through the apex to access the native valve (i.e., progressing in the direction from the left ventricle to the left atrium).

Of course, such minimally-invasive heart valve replacement procedures also pose considerable risk of complications due to embolization, and generally warrant similar preventative measures being taken with adjunctive embolic protection. The most critical anatomical location requiring embolic protection during such procedures is the ascending aorta immediately above the heart, and more particularly with respect to the series of aortic branches located at the aortic arch (i.e., brachiocephalic trunk or innominate artery (BA) which further branches into the right subclavian artery and the right common carotid artery; left common carotid artery (LCA); and left subclavian artery (LSA)). With these aortic branches the primary objective is to prevent embolic debris from entering either the carotid or vertebral arteries and thereby causing neurovascular events.

Previously, it has been proposed to use various tubular filters or curved shields as embolic protection devices within the aorta. Typically, these deflectors are deployed adjacent the internal upper wall of the aortic arch and are positioned to overlie the respective ostium of the aortic branches. Unfortunately, these devices are difficult to deliver, and they may not fully achieve and maintain sufficient apposition with the upper wall of the aortic arch during the interventional procedure. Additionally, such deflectors are susceptible to being dislodged during deployment of valve delivery catheters and prosthetic implants which are being introduced by femoral artery approach. Consequently, these devices might only reduce, but will likely fail to altogether eliminate, the ultimate migration of embolic debris into the aortic branches. It is therefore believed that the most effective and safe embolic protection would be utilizing filters which are directly inserted into the ostium of each aortic branch.

Alternatively, embolic protection devices have been developed for delivery by brachial or radial artery approach. However, these devices require accessing additional patient vasculature in support of a filter delivery already utilizing the femoral artery approach. Statistics indicate that such brachial or radial artery approaches may introduce further complications than the femoral artery approach. Access to the brachial or radial arteries carries not only a higher risk of complications, but the complications are generally more severe than those associated with femoral access. The arteries of the upper extremity have an enveloping fascial sheath. Therefore when a hematoma does occur, brachial plexopathies are more common. In addition, upper extremity vessels tend to spasm more frequently during manipulation, making access more challenging. Brachial access also carries the added risk of distal ischemia and embolization over radial access. Finally, although guiding sheaths up to 6 or 7 French may be percutaneously placed in either vessel, radial access should be preferred over brachial because of a lower complication profile.

While others have previously proposed deployment of multiple embolic filters during cardiac catheterization, with the objective that each aortic branch independently receives an embolic filter, none of these embolic protection systems have been adjunctively sufficient to address all of the following clinical problems associated with TAVI, for example:
Accurate embolic filter delivery and stable deployment within each aortic branch (i.e., embolic filters being firmly deployed within each ostium at the appropriate orientation);
Safe and effective embolic protection for every aortic branch being filtered (i.e., deflectors may not prevent entry of all embolic debris);
Minimal clinical complications by avoiding multiple vascular access sites (i.e., avoiding additional, unnecessary access, such as brachial or radial artery approach, while supporting valve delivery utilizing femoral artery approach);
Presenting minimal structural interference with the therapeutic catheter procedure (i.e., the deployed embolic protective system posing minimal physical obstruction to subsequent delivery of the valve replacement catheter, such as by sequentially deploying multiple, self-locking embolic filters over a single guidewire); and
Ease of retrieval (i.e., a single retrieval catheter capable of retrieving all deployed filters).

It is thus desirable to provide an improved embolic protection system, including delivery and retrieval catheters and associated filter elements, which can provide an accurate and safe deployment and retrieval of multiple embolic filters within the aorta in support of minimally invasive cardiac valve repair and replacement procedures.

### SUMMARY

According to various aspects of the disclosure, a method of deploying multiple filtering elements within selected vasculature includes the steps of delivering and deploying a first filtering element over a primary guidewire to a first vessel branching off a main vessel, locking the first filtering element onto the primary guidewire at a desired location within the first branching vessel, delivering a secondary guidewire to a second vessel branching off the main vessel, delivering a second filtering element over the secondary guidewire to the second branching vessel, deploying the second filtering element at a desired location within the second branching vessel, and locking the second filtering element onto the primary guidewire at the desired location associated with the second branching vessel

According to some aspects, the method of deploying multiple filtering elements may include the steps of removing the secondary guidewire from the second branching vessel, repositioning the secondary guidewire by delivering the secondary guidewire to a third vessel branching off the main vessel, delivering a third filtering element over the secondary guidewire to the third branching vessel, deploying the third filtering element at a desired location within the third branching vessel, and locking the third filtering element onto the primary guidewire at the desired location associated with the third branching vessel.

In according with various aspects of the disclosure, a dual lumen catheter assembly for deploying a filtering device within selected vasculature may include a catheter, a filtering element contained in the filter retaining member, and an activating member coupled between the filter retaining member and the proximal shaft for controllably releasing the filtering element into the selected vasculature. The catheter may include a filter retaining member located at a distal end of the catheter, a guidewire hub connected to a proximal end of the filter retaining member and having a first guidewire lumen for receiving a primary guidewire and a second guidewire lumen for receiving a secondary guidewire, and a proximal shaft connected to a proximal end of the guidewire hub.

According to some aspects, the dual lumen catheter assembly may include a guidewire stop tethered to the filtering element and the guidewire stop includes a collet and locking member for stabilizing the relative longitudinal position of the filtering element. The guidewire stop may be premounted over the primary guidewire before insertion into the guidewire hub of the catheter.

The invention is described in more detail below with reference to the enclosed drawings.

### BRIEF DESCRIPTION OF THE DRAWINGS

In the drawings:

FIG. 1 is a diagrammatic view of a dual-lumen, rapid-exchange catheter according to a first exemplary embodiment of the present disclosure;

FIG. 2 is a cross section view of the catheter along section II-II in FIG. 1;

FIGS. 3A and 3B are diagrammatic views of the distal end of an exemplary embolic filter delivery catheter in accordance with various aspects of the disclosure;

FIGS. 4A and 4B show schematically an exemplary embodiment of a guidewire stop for use with an embolic filter, depicted in an unlocked and locked configuration, according to the disclosure;

FIGS. 5-15 illustrate procedures for delivering, deploying, and retrieving a plurality of filtering devices to aortic branch arteries adjacent the aortic arch;

FIG. 16 shows a femoral introduction of a valve repair assembly according to various aspects of the disclosure.

FIG. 17 shows a femoral introduction of a valve repair assembly and filtering device according to various aspects of the disclosure.

FIG. 18 shows the inflation of a valvuloplasty balloon and emboli being caught by the filtering device according to various aspects of the disclosure.

FIGS. 19 and 20 show a transapical introduction of a valve repair assembly according to various aspects of the disclosure.

FIG. 21 shows a transapical introduction of a valve repair assembly and filtering element according to various aspects of the disclosure.

FIG. 22 shows an implanted prosthetic valve according to various aspects of the disclosure.

FIGS. 23A-23B shows an embolic deflector mesh applied to the upper aortic wall adjacent the aortic branches in accordance with various aspects of the disclosure.

FIG. 24 shows another filtering element that filters the blood entering carotid arteries according to various aspects of the disclosure.

### DETAILED DESCRIPTION

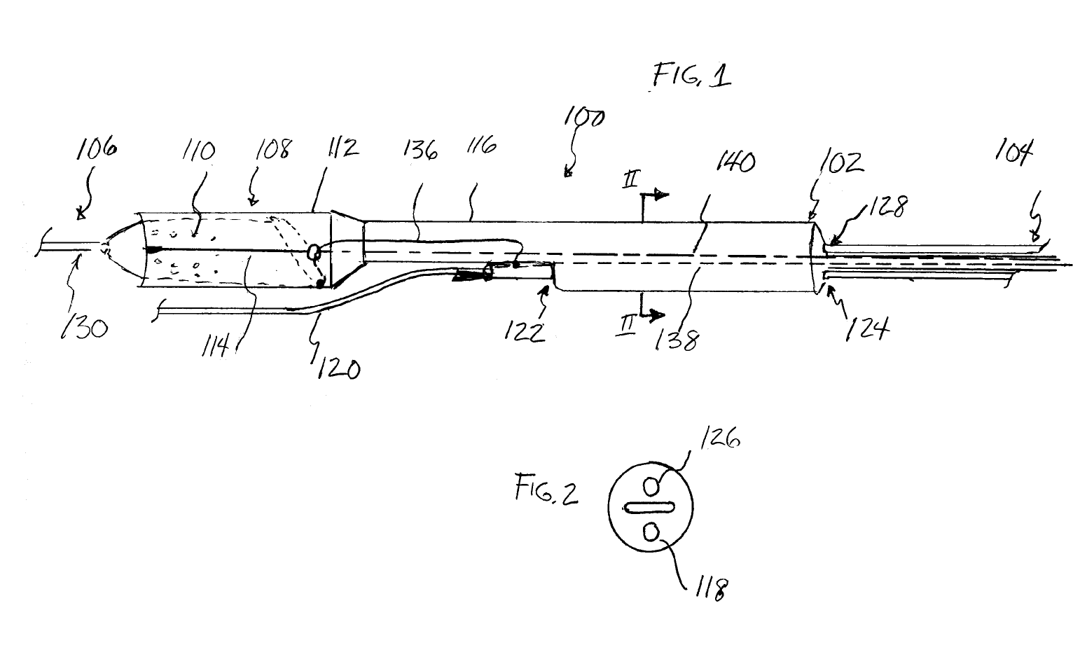

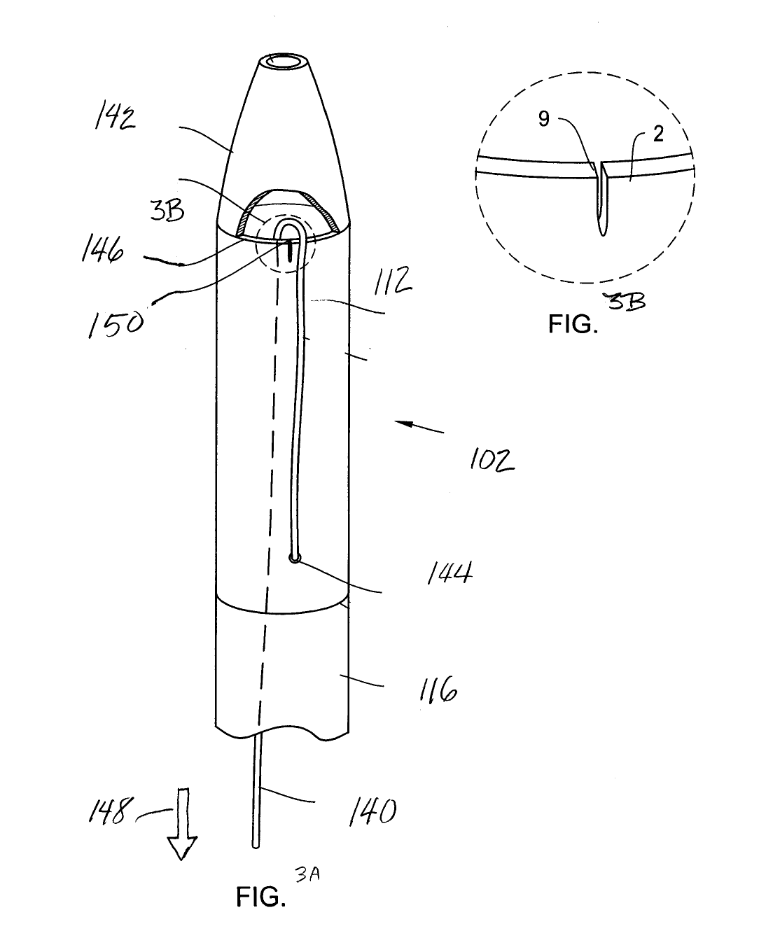

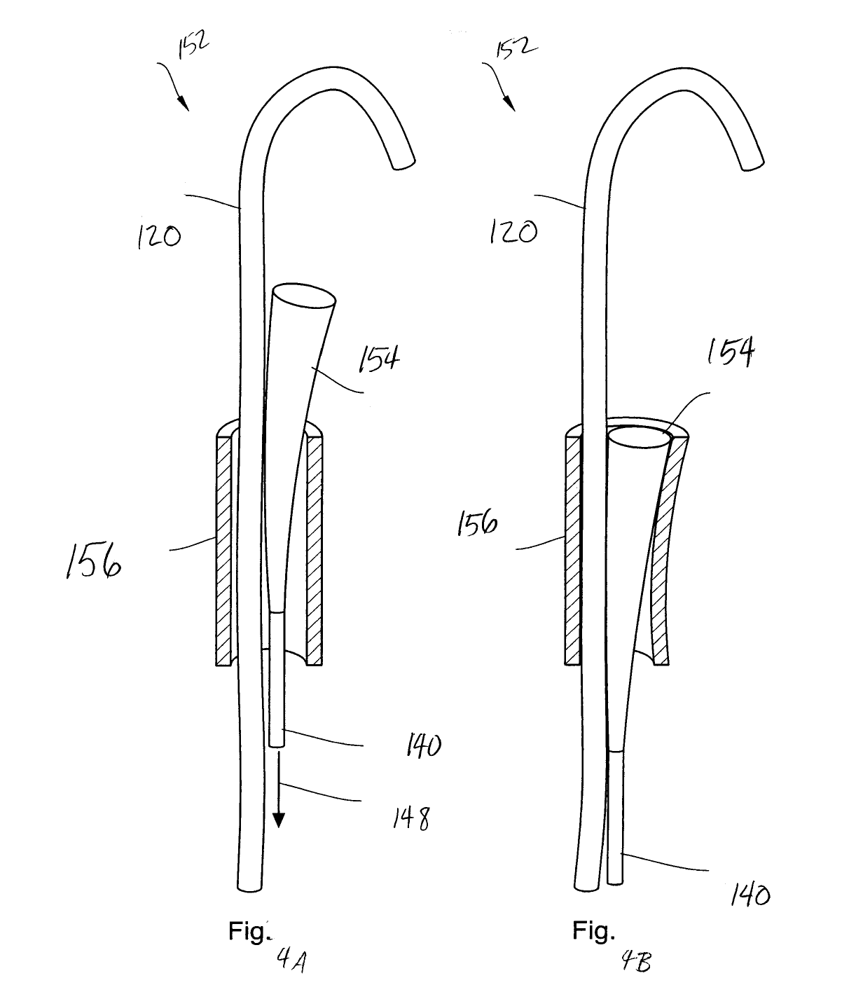

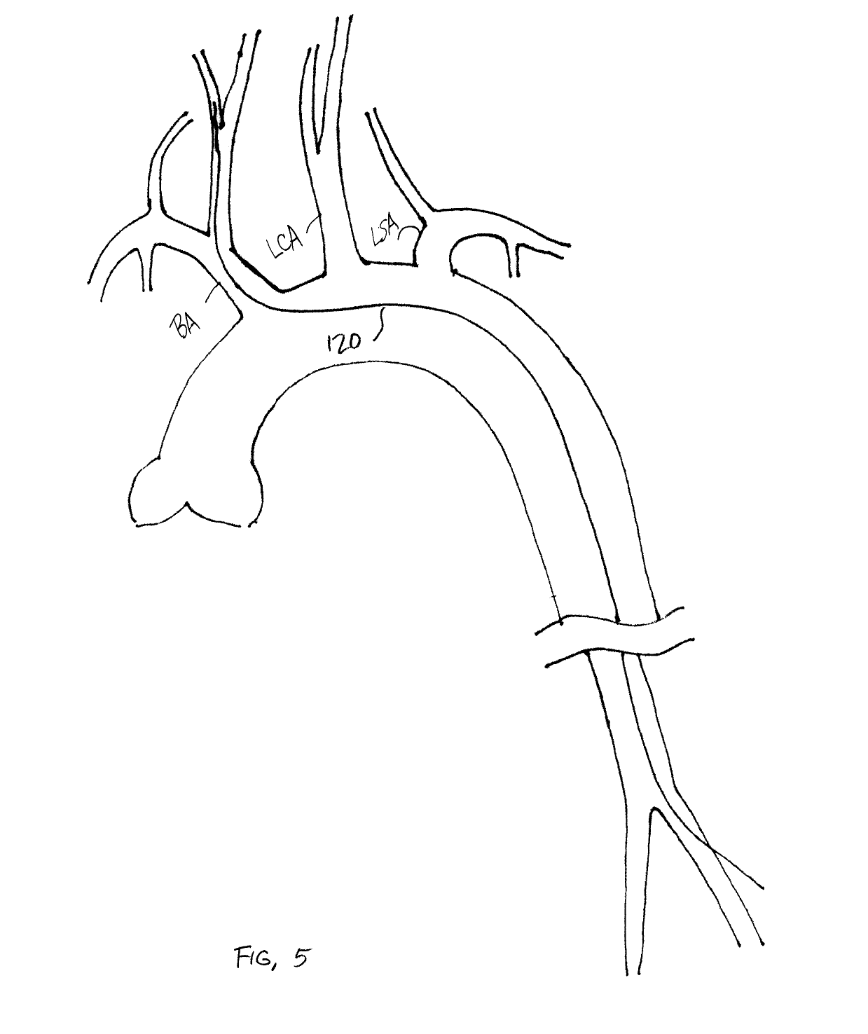

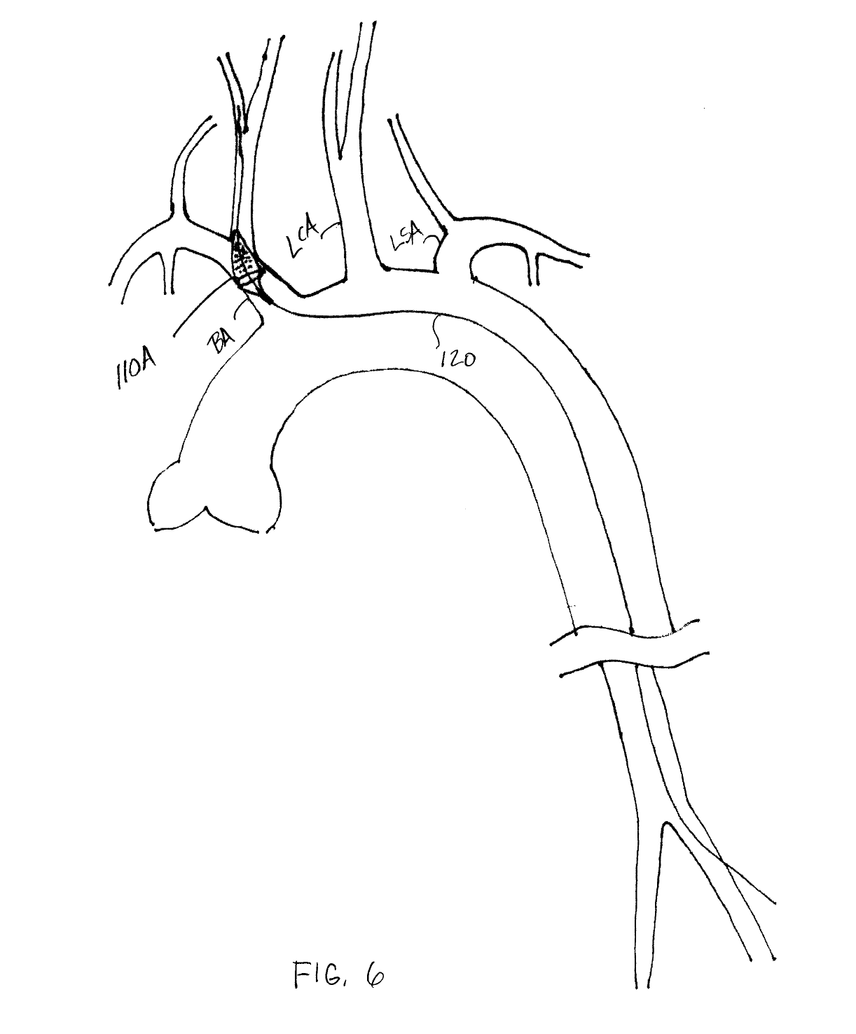

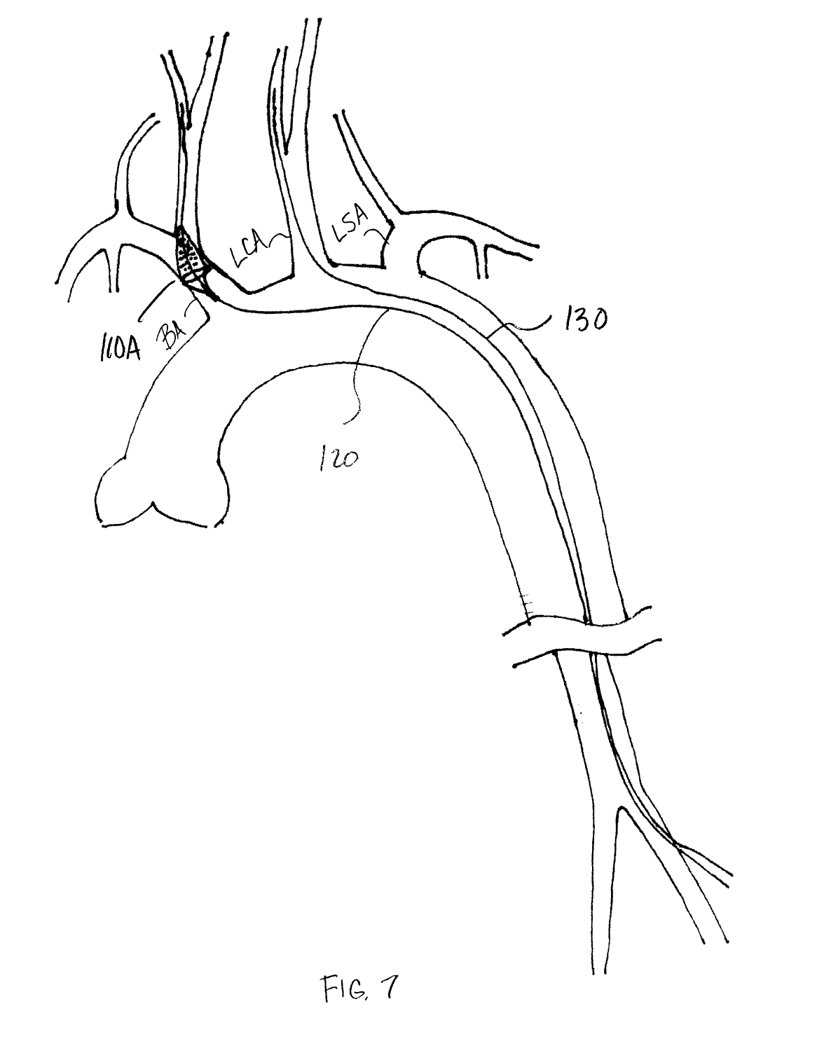

FIG. 1 shows a diagrammatic view of a dual lumen rapid exchange catheter assembly 100 in accordance with various aspects of the disclosure. This delivery catheter is specifically designed to support the delivery of multiple embolic filtering elements which can be deployed and locked in place along a single filter deployment guidewire at desired spaced-apart locations, such as for placement within each ostium for each aortic branch. An example of embolic filters which can be used with this delivery catheter are filters having a locking collet and guidewire locking wedge which are described in more detail below (see, FIGS. 4A-4B).

The catheter assembly 100 includes a catheter 102 having a proximal end 104 and a distal end 106. The distal end 106 of the catheter 102 may include a pod 108 containing a filtering element 110 therein. The pod 108 may comprise a sheath 112 which is designed to split or rupture in a predetermined manner and location 114 (i.e., a split in the sheath which, upon pulling an activation filament, progresses from the distal end to a region proximate the junction between the catheter shaft and pod). Activation of this splittable sheath provides for a controlled release of the filtering element within a desired vascular site (e.g., ostium of an aortic branch being protected). The splittable sheath can comprise a filter constraining sheath which incorporates an activating pulling wire mechanism of the type described in U.S. patent application Ser. No. 12/417,299, filed on Apr. 2, 2009, and entitled “Delivery Catheter with Constraining Sheath and Methods of Deploying Medical Devices into a Body Lumen,” the disclosure of which is incorporated herein by reference.

It will be understood that the catheter can alternatively be provided with a variety of alternative structural designs which provide for controlled release of the filtering device 110 from the pod 108, in lieu of a splittable sheath 112. For example, the pod 108 can be formed as a relatively rigid pod provided with a forward-facing opening. In this case, the filtering element 110 can be released from the pod 108 by advancing a push rod which can be deployed within the guidewire hub extending proximally through the interior of the proximal shaft to urge the filtering element 110 into the desired vascular site.

Extending proximally from the pod 108 is a dual-lumen, rapid-exchange hub 116. A first guide wire lumen 118 is provided within the hub 116, and is configured to receive a primary guide wire 120 that is already deployed to a desired position in a body lumen. In this regard, it is envisioned that the first embolic filter element has already been delivered by femoral artery approach and deployed within the ostium of the aortic branch most proximate to the aortic root (i.e., the brachiocephalic trunk or innominate artery). As such, the original or “primary” guidewire remains attached to the deployed distal filter. Accordingly, this primary guidewire 120 can serve as a deployment platform for subsequently delivered, self-locking embolic filter elements.

As shown in FIGS. 1-2, the first guidewire lumen extends proximally through the hub 116, providing a first distal rapid exchange port 122 and a first proximal rapid exchange port 124 of the hub 116. Also shown in FIGS. 1-2, a second guide wire lumen 126 extends proximally through the pod 108 and the hub 116 and exits at a second proximal rapid exchange port 128 of the hub 116.

As previously indicated, the first guide wire lumen 118 is configured to receive a primary guide wire 120 that is already deployed to a desired position in a body lumen. The second guide wire lumen 126 is configured to receive a secondary guide wire 130 that will be introduced to a desired position in a body lumen which constitutes the next successive location to receive the next filtering element.

Referring to FIGS. 3A and 3B, an exemplary embodiment of the distal end of a delivery catheter 102 in accordance with the present disclosure is shown. FIG. 3A depicts a distal end of the delivery catheter 102. The distal end of delivery catheter 102 may include a tapered tip 142 which may also be formed of relatively soft material which is atraumatic to the vessel being treated. A constraining sheath 112 is attached at the distal end of hub 116 of the catheter 102. The constraining sheath 112 is adapted to receive a therapeutic device, such as an embolic protection element/device (EPD) which is not shown, in a collapsed configuration for later delivery and deployment into the vessel lumen being treated. The EPD can comprise a filter which incorporates a guidewire locking mechanism of the type described in U.S. patent application Ser. No. 11/873,882, filed on Oct. 17, 2007, and entitled “Guidewire Stop,” and U.S. patent application Ser. No. 11/873,893, filed on Oct. 17, 2007, and entitled “Guidewire Stop,” the disclosures of which are incorporated herein by reference.

A pulling wire 140 may be coupled to the catheter sheath 112, for example, at location 144 (FIG. 3A). Pulling wire 140 may be a flexible wire, and may comprise a metal wire or polymer suture, for example. Pulling wire 140 may extend distally from a region 144 of catheter sheath, being secured over the distal rim 146 of the constraining sheath 112. A proximally-extending portion of the pulling wire 140 may extend proximally toward a proximal end of the catheter, with the trailing end of the pulling wire accessible during use for actuation by the interventional vascular practitioner simply pulling the wire 140 in a proximal direction to actuate release of the EPD from delivery sheath 112.

In operation, once the delivery catheter 102 is positioned at the desired vascular treatment site for deployment of the constrained EPD, the trailing portion of the pulling wire 140 is pulled in a proximal direction, as indicated by numeral 148 in FIG. 3A. The resulting pulling motion on wire 140 will produce a longitudinal tear or split of the sheath 112, beginning at the distal location where wire 140 is secured over the distal rim 146. Once the sheath 112 is split to a sufficient degree along its length, as shown for example at split 114 in FIG. 1, the self-expanding EPD (not shown) contained in the sheath 112 is simply released from constraint and permitted to expand into full apposition with the vessel wall just distal of the vascular treatment site.

Since the only forces required for this catheter embodiment to deploy the constrained EPD relate to the proximal force exerted on pulling wire 140 to effectuate a longitudinal split or tear in sheath 112, deployment can be accomplished with relative ease. This EPD deployment approach, for example, entirely avoids the necessity of overcoming frictional forces associated with relative longitudinal movement between a constrained EPD and a constraining sheath.

As a further improvement to this embodiment, for example, a relatively small cut or preformed tear zone 150 can be provided in the sheath 112 adjacent the distal rim 146, to facilitate splitting of the sheath at a desired location. According to further aspects of the invention, the pulling wire 140 may include a cutting edge, or incorporate abrasive materials, such as diamond dust, in order to facilitate tearing of the sheath 112.

Referring now to FIGS. 4A and 4B, the guidewire stop 152 which is used to physically lock the EPD onto the primary guidewire 120 is illustrated, depicting an unlocked configuration (FIG. 4A) and a locked configuration (FIG. 4B). In this embodiment, the guide wire locking element 154 is implemented as a tapered (wedge-shaped) element 154, which is urged into locking engagement with a guide wire locking collet 156 (FIG. 4B) by activating the pulling wire 140 by retracting the wire in a proximal direction as shown by arrow 148.

As shown in FIGS. 4A-4B, the guidewire stop assembly 152 may be disposed on the primary guide wire 120. The filtering device 110 may be coupled to the collet 156 via a tether 136 (FIG. 1), and a first pull wire 138 may extend proximally from the collet 156. Thus, activating the guidewire stop will wedge and physically engage the stop between the locking collet 156 and the primary guidewire 120. The tether 136 thereby prevents the EPD from undergoing any significant lateral movement relative to the guidewire stop 152.

Advantageously, locking collet 156 may be formed from a springy or yielding material to allow for slight deformation or expansion of locking collet 156 when the wedge-shaped element 154 is drawn into the locking tube 156, as indicated in FIG. 4B. This allows the locking element 154 to be more securely locked on primary guidewire 120. The locking tube 156 may be formed, for example, from stainless steel, nitinol, plastic, or any other material exhibiting an appropriate degree of springiness or elasticity.

FIGS. 5-12 illustrate the method of sequential introduction and deployment of multiple embolic filter elements/devices 110A (first EPD), 110B (second EPD), 110C (third EPD) into the abovementioned aortic branches. Each introduction is accomplished by use of an independent guidewire, and all introductions are by the femoral artery approach.

The first EPD device 110A is delivered over a pre-advanced guidewire 120, which has already been introduced in bare guidewire fashion to the desired vascular site (i.e., brachiocephalic trunk or innominate artery—BA), as shown in FIG. 5. In particular, this first EPD device 110A is delivered by a filter delivery catheter which is equipped with an activating pulling wire to activate the guidewire stop mechanism 152 hereinabove described. Once the initial EPD element 110A has been locked onto the initial guidewire 120 (FIG. 6), this guidewire becomes the “primary” guidewire to which subsequently delivered EPD devices 110B and 110C are engaged and locked into position.

However, the delivery of the subsequent EPD filters 110B and 110C is accomplished by using the dual-lumen, rapid-exchange catheter 102, in conjunction with a primary guidewire 120 and a secondary guidewire 130. FIG. 7 illustrates the distal end of the primary guidewire 120 being introduced into the brachiocephalic trunk or innominate artery (BA), with the first EPD filter 110A locked into place within the ostium of this aortic branch. FIG. 7 also illustrates the distal end of a secondary guidewire 130 being introduced into the next adjacent aortic branch (i.e., left common carotid artery—LCA), and is ready to receive the second EPD filter.

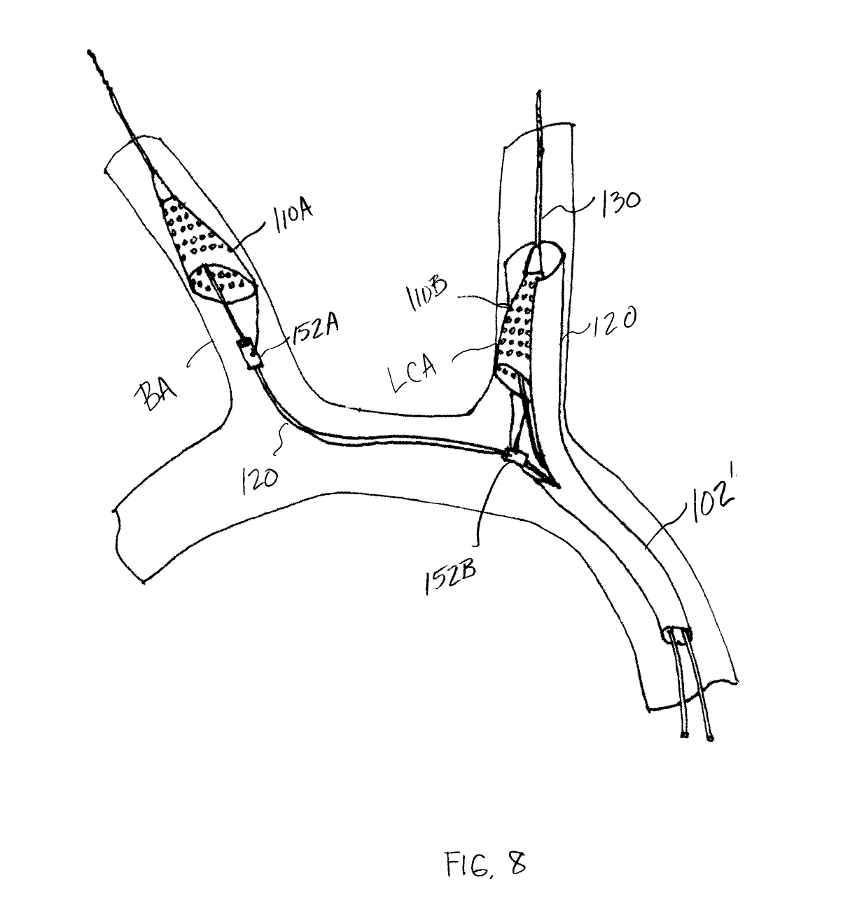

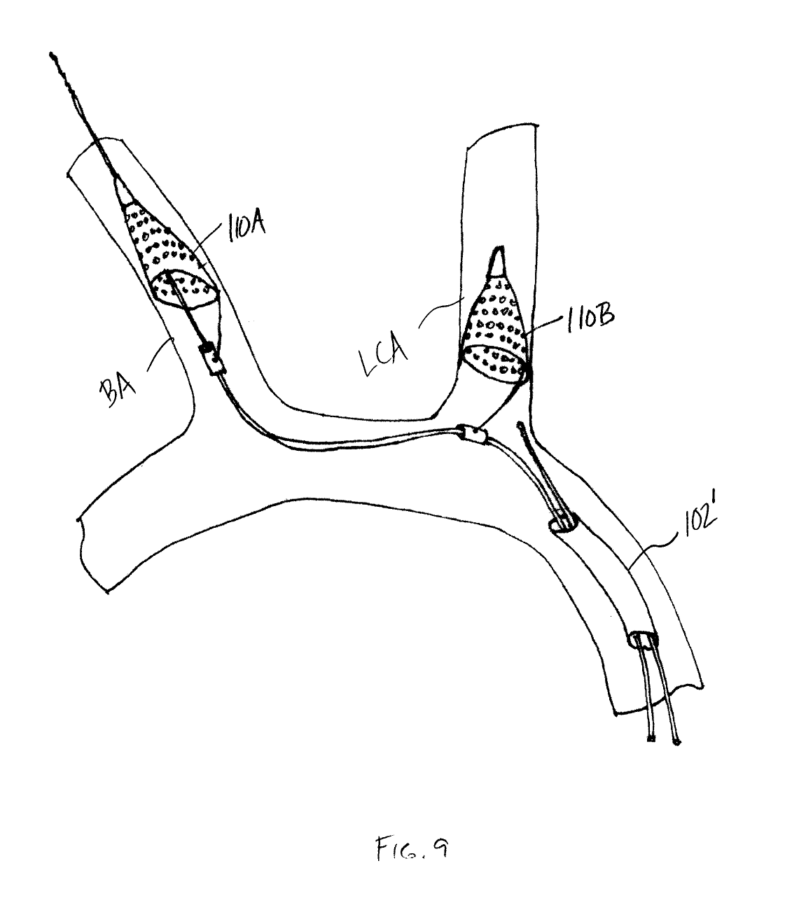

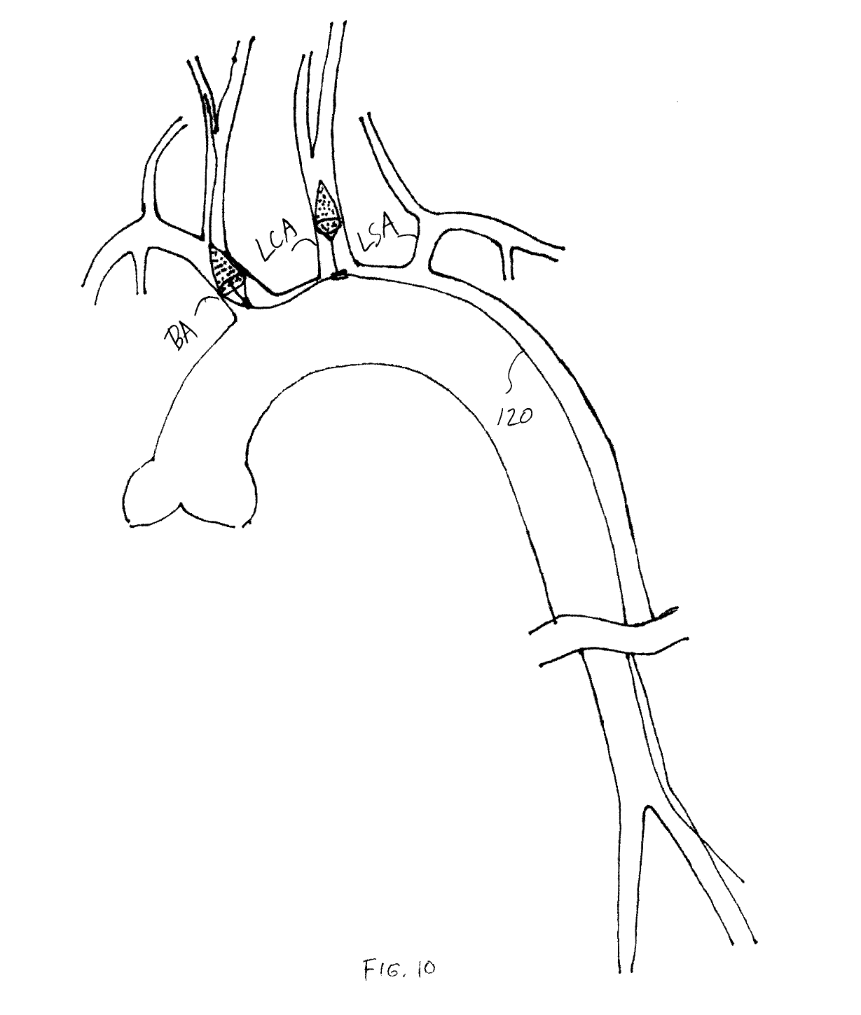

FIG. 8 illustrates the delivery and deployment of the first and second EPD devices 110A and 110B, which are shown tethered to their respective guidewire stops 152a and 152B, following activation and locking engagement with the primary guidewire 120 at the desired locations (i.e., adjacent the BA and LCA ostia). The details of the filter delivery catheter are only shown schematically at 102′ but will be readily understood to correspond to the structure hereinabove described with respect to catheter 102 in FIG. 1. During the delivery procedure for the second EPD filter 110B, a secondary guidewire 130 is first introduced in bare guidewire manner into the next adjacent aortic branch (LCA), where it temporarily remains for the purpose of introducing the filter over it (FIG. 8).

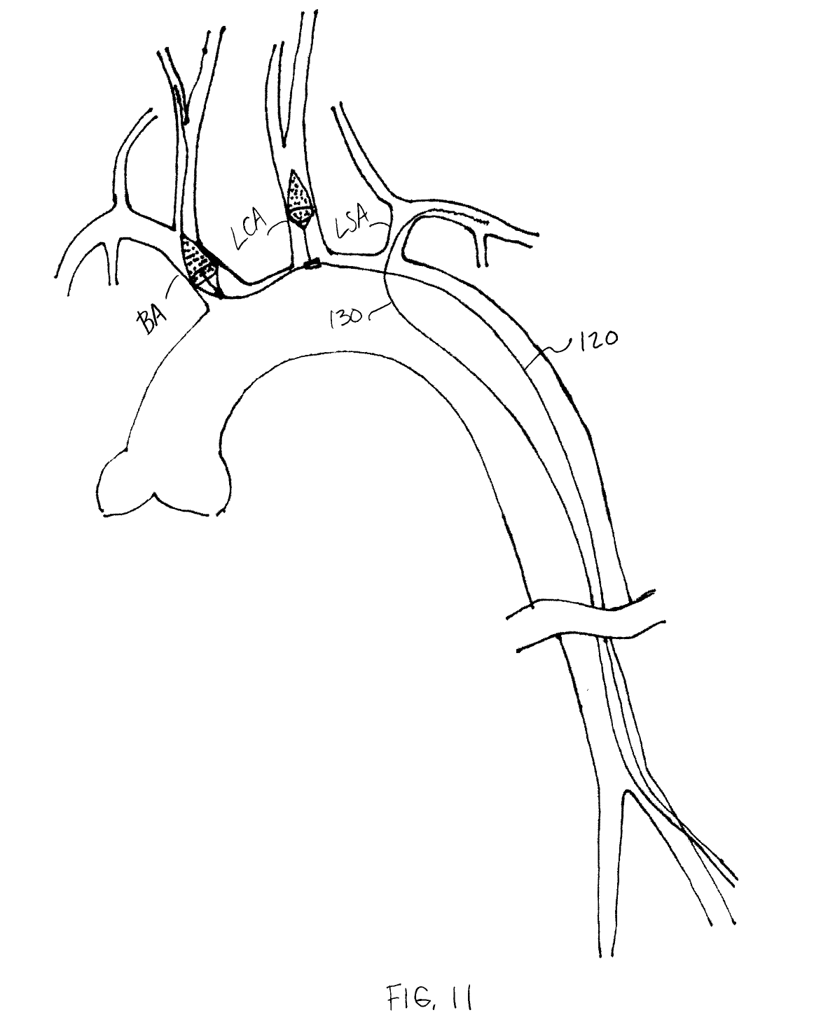

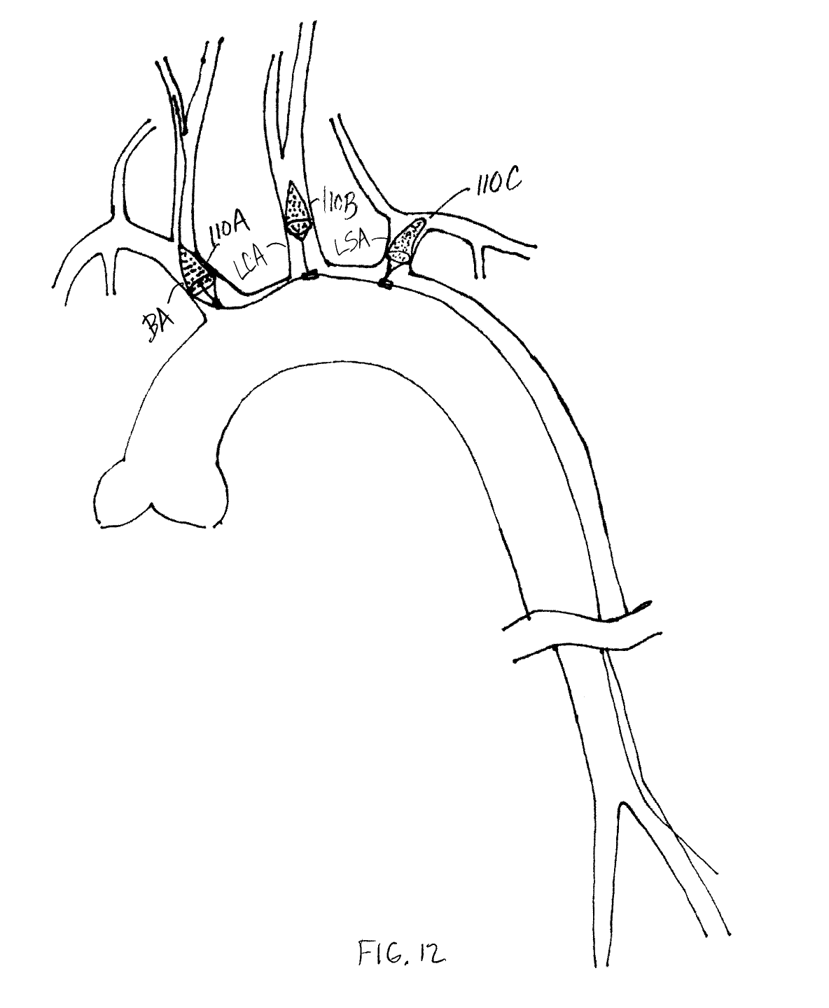

As illustrated in FIG. 9, following activation of the pulling wire and splitting of the constraining sheath 120, the second EPD filter is suitably released and deployed within the ostium of the LCA. As illustrated in FIGS. 9-10, following deployment of the second EPD filter, the secondary guidewire 130 is withdrawn from the vasculature, while leaving the primary guidewire 120 in place to provide a stable anchoring location for the deployed EPD filters 110A and 110B.

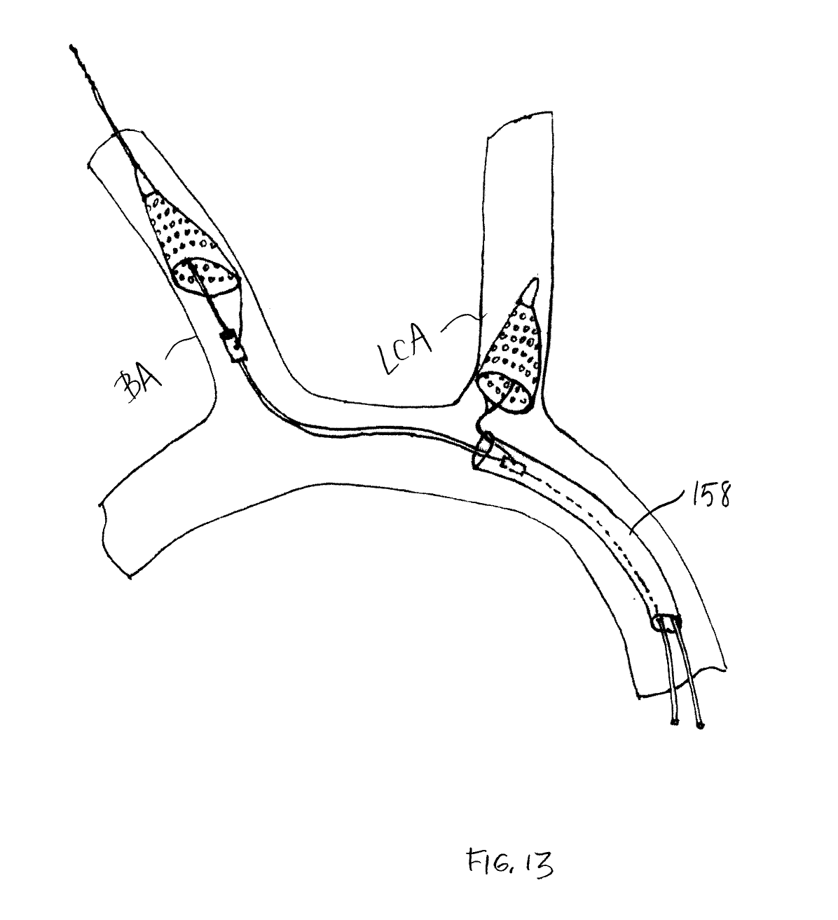

FIGS. 11-12 illustrate introduction of a secondary guidewire 130 into the next adjacent aortic branch (i.e., left subclavian artery—LSA), and a successful deployment of a third EPD filter within the ostium of the LSA. At this point, all three of the aortic branches are properly protected from embolic debris which might be generated during any interventional valve procedure, such as TAVI. Advantageously, however, the ability to deploy and locked each tethered EPD filter along a single, primary guidewire 120 results in minimal obstruction to the process of introducing a the appropriate valve delivery catheter through the same aortic region, such as would occur using the femoral artery approach.

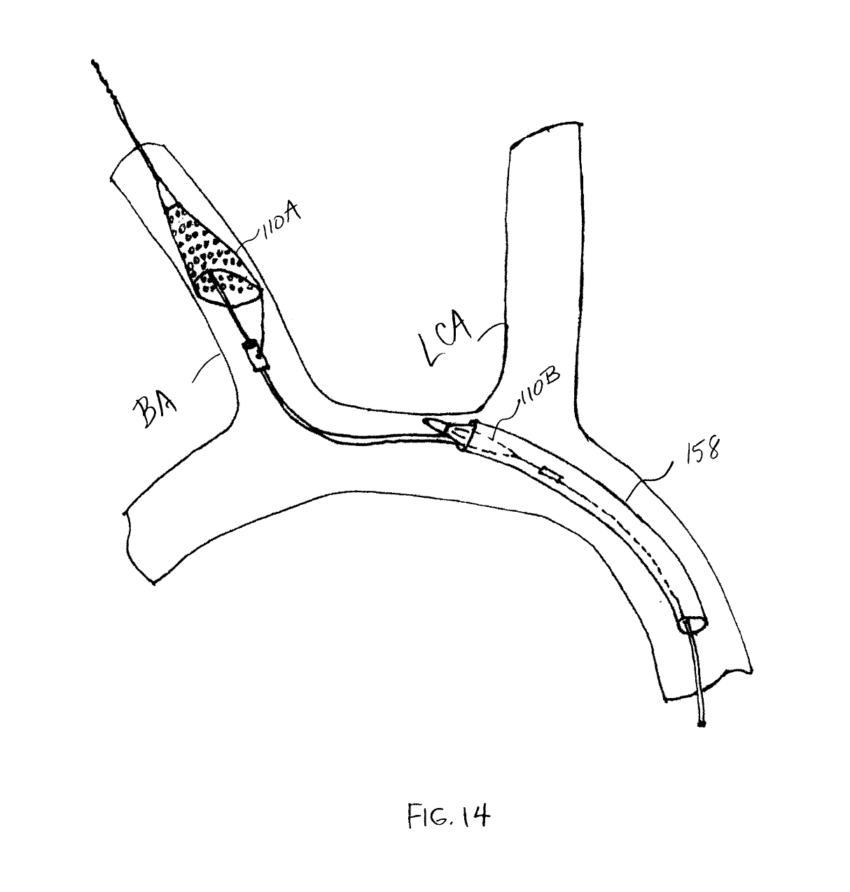

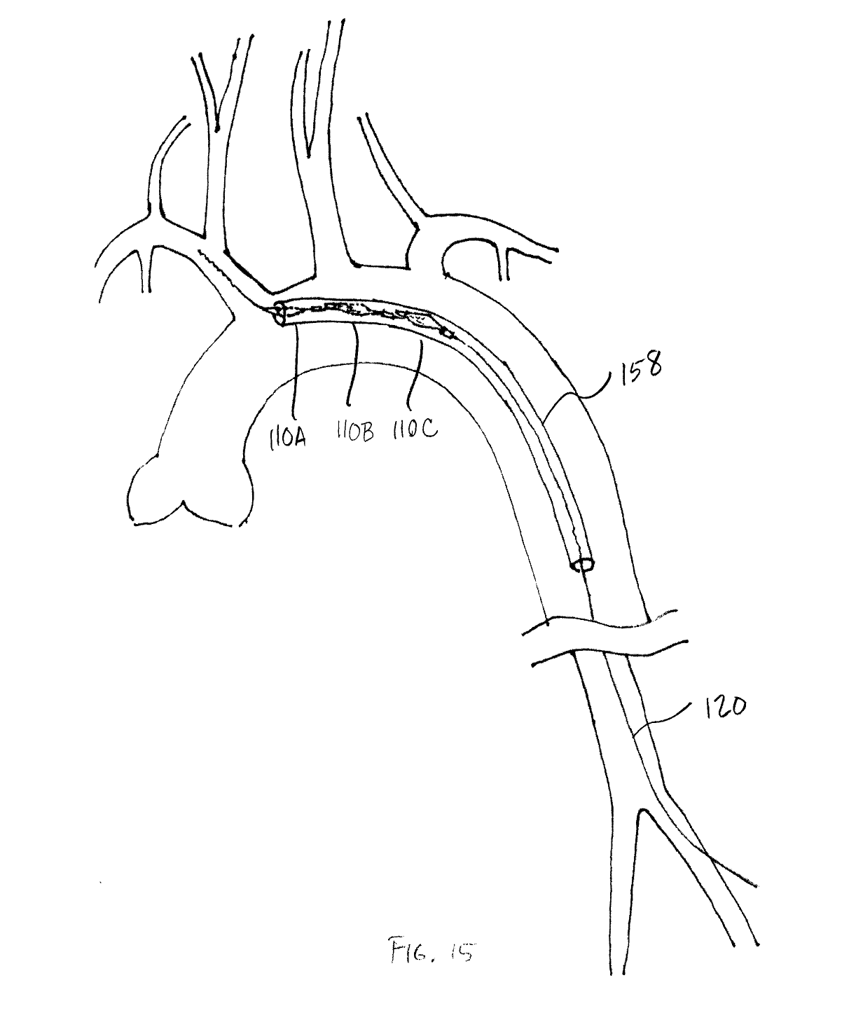

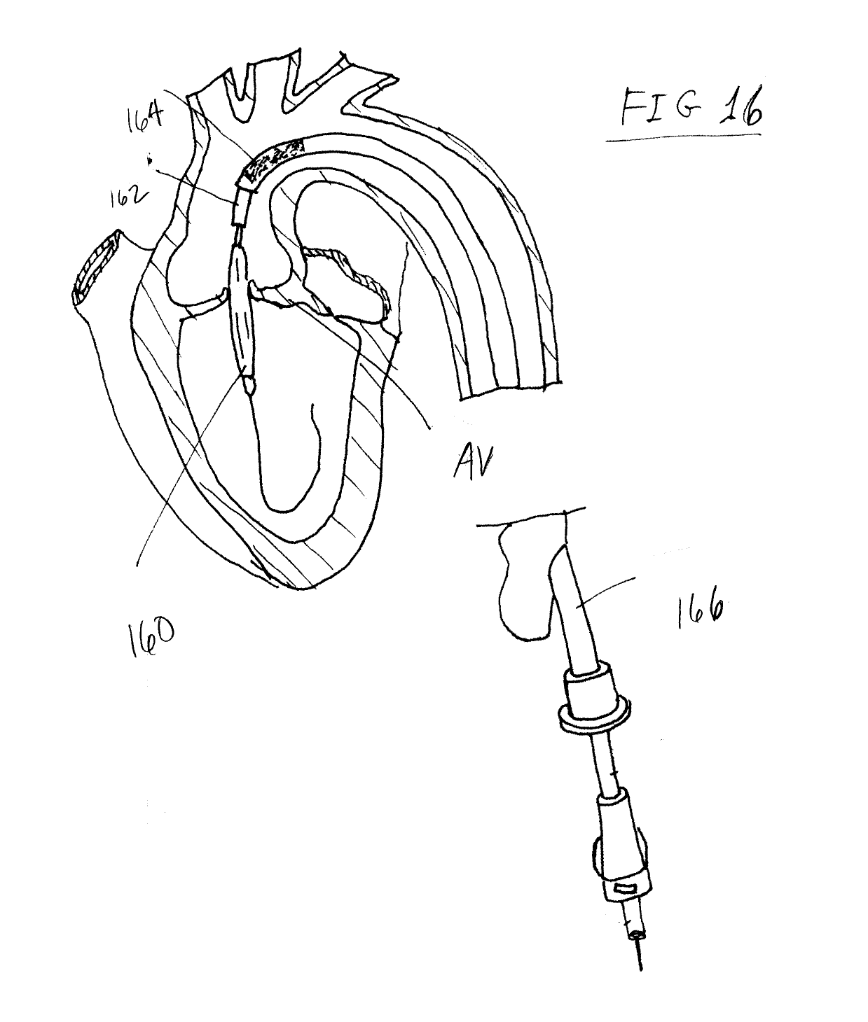

FIGS. 13-15 illustrate retrieval of the multiple EPD filters 110A, 1108 and 110C. Since the EPD filters are independently locked onto a single, primary guidewire 120, it is possible to retrieve all of these embolic filters by advancement of a single retrieval catheter 158 over the filters. Following collapse and containment of all of these filters, the guide catheter 158 and primary guidewire are proximally withdrawn from the patient's vasculature as a single unit.

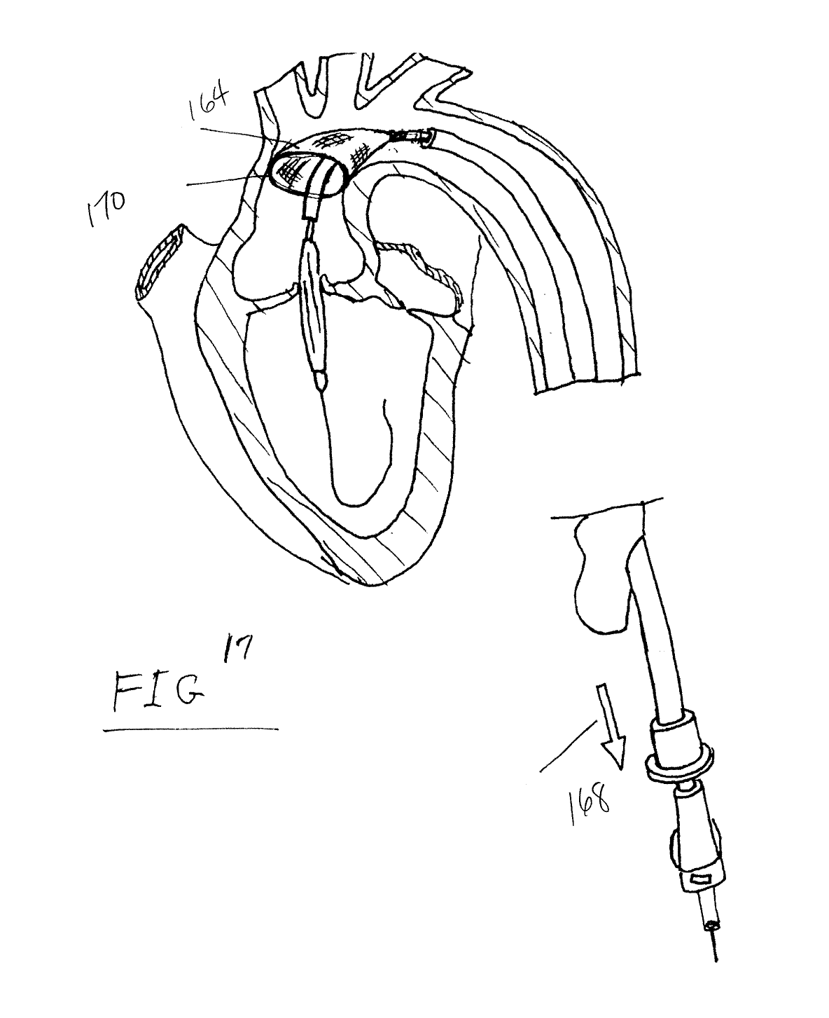

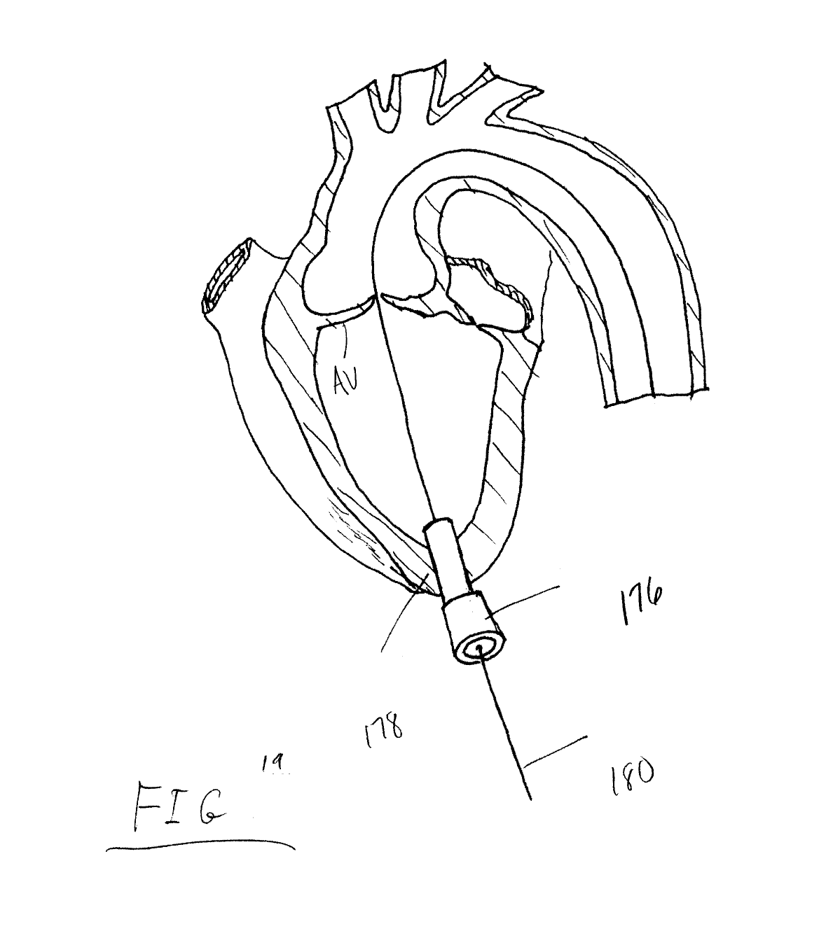

FIGS. 16-18 illustrate introduction of a valvuloplasty balloon catheter 160 for treatment of a diseased aortic valve (AV). In FIG. 16, a guide catheter 162 is deployed by femoral artery introduction, traversing the aortic arch, and being positioned at the base of the ascending aorta. The valvuloplasty catheter 160 is introduced through the guide catheter 162 until the balloon element is positioned across the aortic valve (AV). The distal end of the guide catheter incorporates a self-expanding embolic filter element 164 which is constrained in a collapsed configuration by constraint provided by an over tube 166.

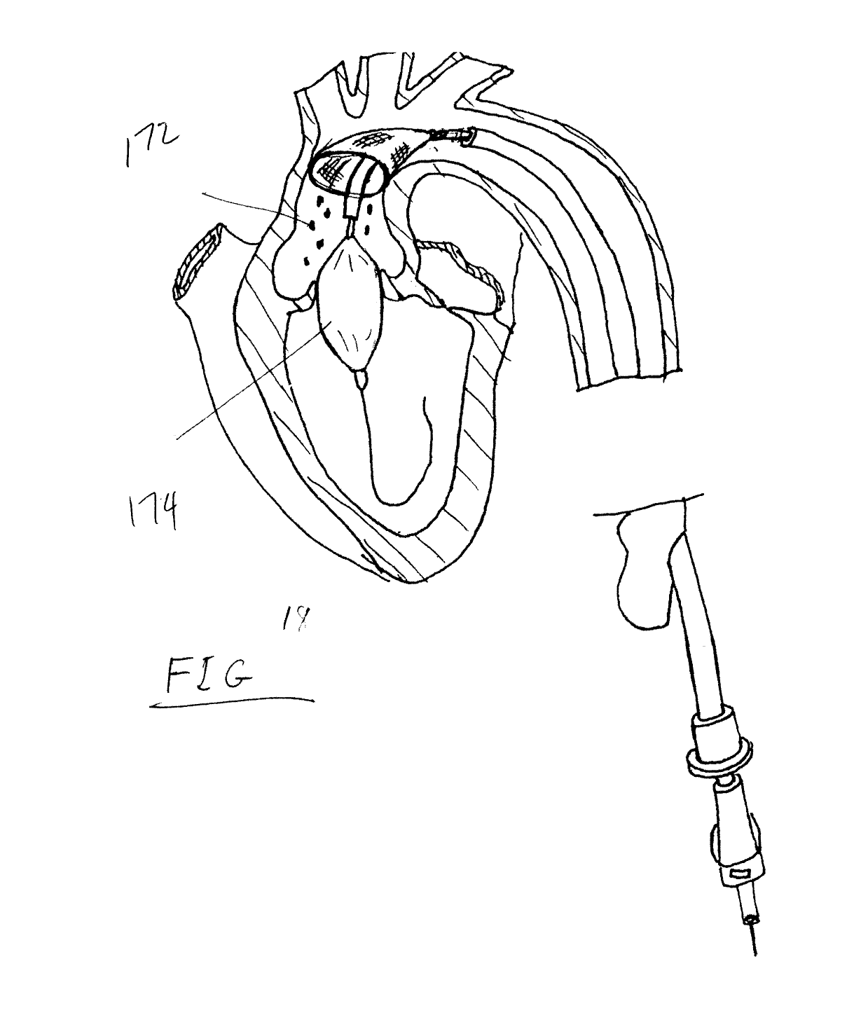

In FIG. 17, the embolic filter element is allowed to expand into an opened configuration by proximal retraction of the over tube 166 in the direction of arrow 168. Consequently, the distal rim 170 of the filter element 164 is deployed in sufficient wall apposition with the aorta to provide embolic protection with respect to embolic debris 172 potentially generated during the valvuloplasty procedure by the balloon element 174. In this example, the filter is a mesh filter that braided or knitted from Nitinol or cobalt-chromium wires. Alternatively, this mesh can be fabricated from a polymeric membrane with holes drilled in it, possibly by laser ablation or other drilling method. Examples of suitable polymers comprise Nylon 11 or Nylon 12, as well as polyurethane, PTFE or other similar materials. In this case it may also be desirable to incorporate frame formed of metallic or plastic construction to help to retain its deployed shape and facilitate in filter folding and retrieval.

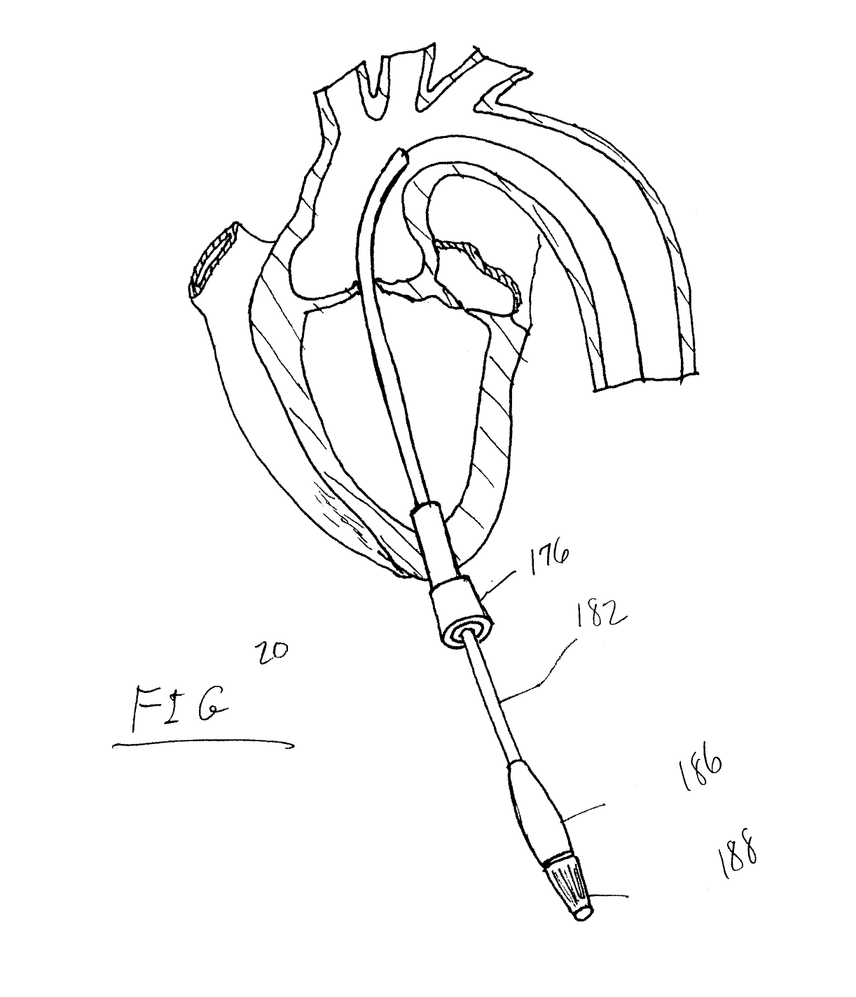

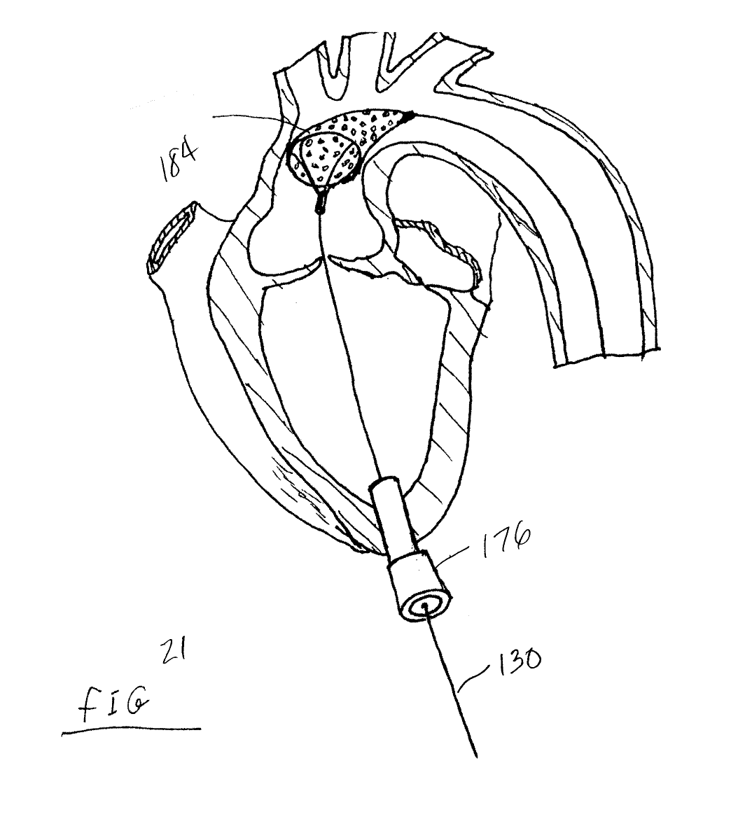

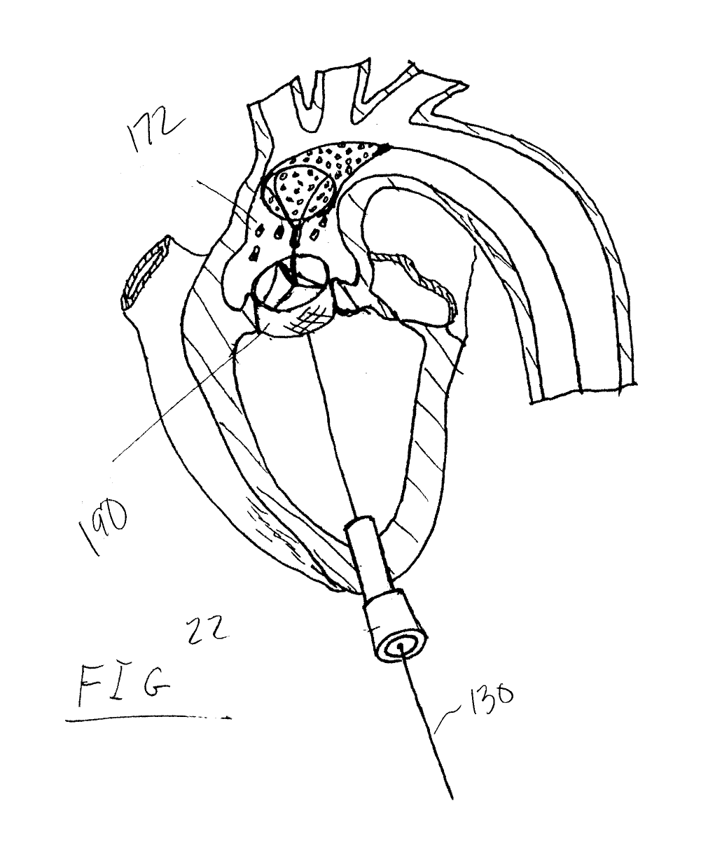

FIGS. 19-22 illustrate transapical introduction of a replacement valve. As depicted in FIG. 19, an insertion sheath 176 is introduced into the apex 178 of the heart. Subsequently, a guidewire 180 is introduced through the insertion sheath 176, and the distal end of the guidewire 180 is passed though the diseased aortic valve (AV), around the aortic arch and into the descending aorta. In FIG. 20, a filter delivery catheter 182 which carries an embolic filter element 184 constrained in a collapsed configuration within the distal end of the catheter sheath, is introduced over the pre-advanced guidewire 180 and through the insertion sheath 176. The distal end of the sheath 182 is positioned on the opposite side of the aortic valve, so that the filter can be released and deployed within the ascending aorta to capture embolic debris 172 subsequently generated during implantation of a replacement valve. The delivery catheter 182 is provided with an activating handle 186 and rotational knob 188 cooperate to controllably release the embolic filter upon demand. If desired, the embolic filter can be locked anywhere on the wire, and it can also be detached and re-attached in a new location. In FIG. 22, the prosthetic replacement valve 190 is shown being deployed in the location corresponding to the native aortic valve, and the embolic debris 172 is being captured by the filter element 184.

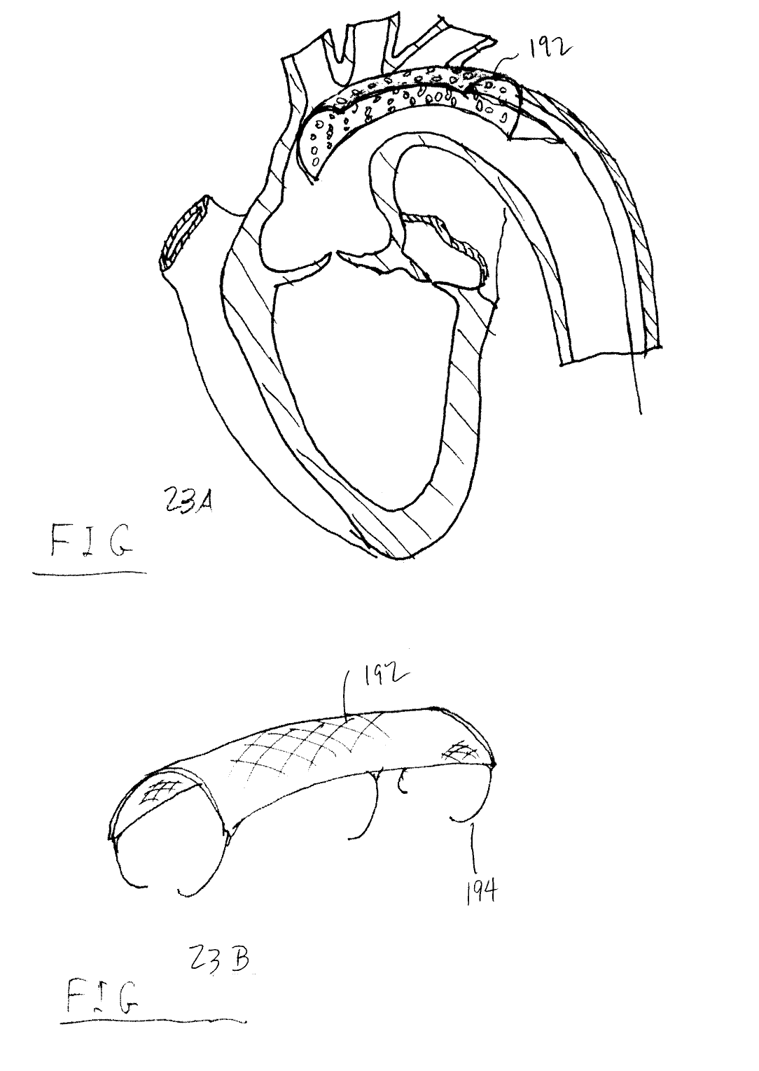

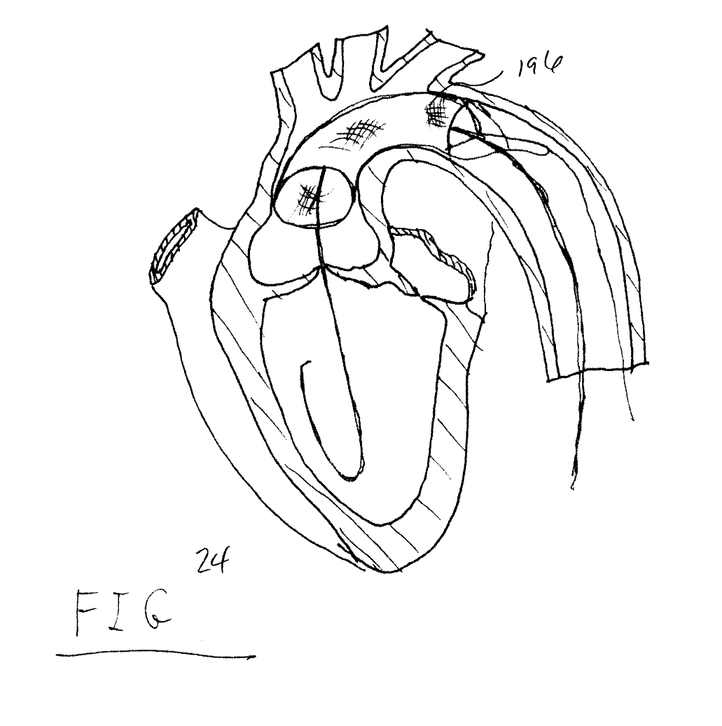

FIGS. 23A-23B depicts another approach for protecting the aortic branches. With this embodiment, an embolic deflector 192 formed of a filtering net or mesh-like membrane is applied to the upper internal wall of the aortic arch. While embolic deflectors are generally known, the embolic deflector 192 depicted in FIG. 23B is provided with stabilizing struts 194 formed into semicircular configurations which project downwardly from the deflector base to rest against the aortic wall on the opposite side of the filter, thereby improving the stability of the deployed deflector filter during use to ensure effective wall apposition and filtration at the ostia of the aortic branches.

FIG. 24 shows another filter device 196 which filters the blood entering carotid arteries, and comprises a closed tubular structure. This structure is designed to permit insertion of therapeutic catheters therethrough with minimal obstruction or interference with the procedures.

It will be apparent to those skilled in the art that various modifications and variations can be made to the arterial filters, catheters or arterial filters, and methods of deploying same of the present disclosure without departing from the scope of the invention. Throughout the disclosure, use of the terms “a,” “an,” and “the” may include one or more of the elements to which they refer. Other embodiments of the invention will be apparent to those skilled in the art from consideration of the specification and practice of the invention disclosed herein. It is intended that the specification and examples be considered as exemplary only.

What is claimed is:

1. A method of deploying multiple filtering elements within selected vasculature, comprising the steps of:
a) delivering and deploying a first filtering element over a primary guidewire to a first vessel branching off a main vessel;
b) locking the first filtering element onto the primary guidewire at a desired location within the first branching vessel;
c) delivering a secondary guidewire to a second vessel branching off the main vessel;
d) delivering a second filtering element over the secondary guidewire to the second branching vessel;
e) deploying the second filtering element at a desired location within the second branching vessel; and
f) locking the second filtering element onto the primary guidewire at the desired location associated with the second branching vessel.

2. The method of claim 1 further comprising the steps of:
a) removing the secondary guidewire from the second branching vessel;
b) repositioning the secondary guidewire by delivering the secondary guidewire to a third vessel branching off the main vessel;
c) delivering a third filtering element over the secondary guidewire to the third branching vessel;
d) deploying the third filtering element at a desired location within the third branching vessel; and
e) locking the third filtering element onto the primary guidewire at the desired location associated with the third branching vessel.

3. A dual lumen catheter assembly for deploying a filtering device within selected vasculature, comprising:
a catheter having
a filter retaining member located at a distal end of the catheter,
a guidewire hub connected to a proximal end of the filter retaining member and having a first guidewire lumen for receiving a primary guidewire and a second guidewire lumen for receiving a secondary guidewire, and
a proximal shaft connected to a proximal end of the guidewire hub;
a filtering element contained in the filter retaining member; and
an activating member coupled between the filter retaining member and the proximal shaft for controllably releasing the filtering element into the selected vasculature.

4. The dual lumen catheter assembly of claim 3, further comprising:
a guidewire stop tethered to the filtering element and the guidewire stop includes a collet and locking member for stabilizing the relative longitudinal position of the filtering element,
wherein the guidewire stop is premounted over the primary guidewire before insertion into the guidewire hub of the catheter.
